(19) United States
(12) Patent Application Publication
(10) Pub. No.: US 2025/0082571 A1
(43) Pub. Date: Mar. 13, 2025

(54) COMPOSITIONS AND METHODS FOR DELIVERY OF IL-2 AND ANTI-IL-2 FOR TREATMENT OF RHEUMATOID ARTHRITIS

(71) Applicants: Arizona Board of Regents on behalf of Arizona State University, Scottsdale, AZ (US); Arizona Board of Regents on Behalf of the University of Arizona, Tucson, AZ (US)

(72) Inventors: Abhinav Acharya, Tempe, AZ (US); Hsin-Jung (Joyce) Wu, Columbus, OH (US)

(21) Appl. No.: 18/721,326

(22) PCT Filed: Dec. 21, 2022
(86) PCT No.: PCT/US2022/082123

§ 371 Date: Jun. 18, 2024

Related U.S. Application Data
(60) Provisional application No. 63/293,252, filed on Dec. 23, 2021.

(51) Int. Cl. A61K 9/06; A61K 38/20; A61K 47/34; A61P 37/06; C07K 16/24

(52) U.S. Cl. CPC A61K 9/06; A61K 38/2013; A61K 47/34; A61P 37/06; C07K 16/246

(57) ABSTRACT

The present invention provides a sustained drug release system comprising IL-2 and anti-IL-2 incorporated into a thermosensitive hydrogel that is engineered to be in liquid form at low temperatures for delivery, but form a hydrogel at physiological body temperatures to act as a sustained release platform. The thermostable hydrogel solution slowly releases IL-2 and anti-IL-2 to provide optimal concentrations for effective treatment of autoimmune diseases.

### CROSS-REFERENCE TO RELATED APPLICATIONS

This application claims priority to U.S. Provisional Patent Application No. 63/293,252, filed on Dec. 23, 2021, the contents of which are incorporated by reference herein in its entirety.

### STATEMENT REGARDING FEDERALLY SPONSORED RESEARCH OR DEVELOPMENT

This invention was made with government support under R01 AR078343, R01 HL148347, and R01 AI107117 awarded by the National Institutes of Health. The government has certain rights in the invention.

### BACKGROUND OF THE INVENTION

Autoimmune diseases have risen at a steep rate in the industrialized world and emerge as new epidemics (Bach, J. F. (2002). N Engl J Med 347, 911-920. 10.1056/NEJMra020100; Lerner, A., Jeremias, P., and Matthias, T. (2021). International Journal of Celiac Disease 3, 151-155; Sprouse, M. L., Bates, N. A., Felix, K. M., and Wu, H. J. (2019). Immunology 156, 305-318. 10.1111/imm.13037). Gut microbiota have been implicated in the development of many autoimmune diseases and murine disease models (Ruff, W. E., Greiling, T. M., and Kriegel, M. A. (2020). Nat Rev Microbiol 18, 521-538. 10.1038/s41579-020-0367-2). However, how the gut microbiota regulate disease outside the gut remains poorly understood. Autoantibodies (auto-Abs) play critical roles in the pathogenesis of autoantibody-mediated autoimmune diseases such as rheumatoid arthritis and lupus (Chang, M. H., and Nigrovic, P. A. (2019). JCI Insight 4. 10.1172/jci.insight.125278; Huijbers, M. G., Plomp, J. J., van der Maarel, S. M., and Verschuuren, J. J. (2018). Annals of the New York Academy of Sciences 1413, 92-103. https://doi.org/10.1111/nyas.13561; Suurmond, J., and Diamond, B. (2015). J Clin Invest 125, 2194-2202. 10.1172/jci78084). T follicular helper (Tfh) cells that specialize in B cell help are a subset of CD4⁺ T cells co-expressing high levels of the inhibitory co-receptor PD-1 and the chemokine receptor CXCR5 (Crotty, S. (2011). Annu Rev Immunol 29, 621-663. 10.1146/annurev-immunol-031210-101400; Ma, C. S., Deenick, E. K., Batten, M., and Tangye, S. G. (2012). J Exp Med 209, 1241-1253. 10.1084/jem.20120994). Tfh cells promote the germinal center B cell responses that in turn help the production of high-titer, high-affinity, isotype-switched Abs. Therefore, an excessive Tfh cell response can lead to over-productive auto-Ab responses and autoimmune conditions (Ueno, H., Banchereau, J., and Vinuesa, C. G. (2015). Nat Immunol 16, 142-152. 10.1038/ni.3054).

IL-2 and anti-IL-2 complexes are known to cause robust proliferation of regulatory T cells in vivo. However, due to soluble nature of these complexes and fast diffusion kinetics these need to be provided highly frequently to see the desired effect. Due to their fast turnover, current IL-2 and anti-IL-2 need to be provided every day or every other day to see anti-inflammatory responses in diseases such as rheumatoid arthritis (RA), type-1 diabetes (T1D), idiopathic inflammatory myopathies, multiple sclerosis, traumatic brain injury, wound healing, inflammatory diseases, pemphigus, psoriasis, psoriatic arthritis, systemic lupus erythematosus, ankylosing spondylitis, polymyositis, dermatomyositis, Sjogren's syndrome and different forms of vasculitis.

There is thus a need in the art for compositions and methods for stable, sustained delivery of IL-2 and anti-IL-2 complexes. The present invention addresses this unmet need in the art.

### SUMMARY OF THE INVENTION

In one embodiment, the invention relates to a thermosensitive hydrogel composition comprising:
a) at least one biologically compatible polymer; and
b) at least one of: an IL-2 polypeptide component, an anti-IL-2 antibody component and a combination thereof, wherein the composition is liquid at or below room temperature but forms a gel at about 37° C.

In one embodiment, the thermosensitive hydrogel comprises poly(D,L-lactide-co-glycolide) (PLGA)-poly(ethylene glycol) (PEG)-(PLGA); poly(lactic acid) (PLA)-(PEG)-(PLA); or poly(lactide-co-caprolactone) (PLCL)-(PEG)-(PLCL).

In one embodiment, the thermosensitive hydrogel comprises at least one polyurethane based polymer.

In one embodiment, the IL-2 polypeptide component is an IL-2 polypeptide, a recombinant IL-2 polypeptide, or an active IL-2 polypeptide fragment.

In one embodiment, the anti-IL-2 antibody component is JES6-1, JES6-1A12, S4B6, MQ1-17H12, 2F11C5, 2H20L7, C5, 8D5, BG5, OTI2C9, 5334, 5335, OT15A9, 419A7A3, AB12-3G4, 297C16G2, 297C16F11, 1C5, 1C1, 62, or 3C8.

In one embodiment, the composition comprises a combination of an IL-2 polypeptide and an anti-IL-2 antibody.

In one embodiment, the composition comprises an IL-2 antigen:anti-IL-2 complex or a molecule resembling an IL-2 antigen:anti-IL-2 complex.

In one embodiment, the composition is a low viscosity liquid at room temperature.

In one embodiment, the invention relates to a method of treating a disease or disorder, the method comprising administering an effective amount of at least one thermosensitive hydrogel composition comprising:
a) at least one biologically compatible polymer; and
b) at least one of: an IL-2 polypeptide component, an anti-IL-2 antibody component and a combination thereof, wherein the composition is liquid at or below room temperature but forms a gel at about 37° C.

In one embodiment, the thermosensitive hydrogel comprises poly(D,L-lactide-co-glycolide) (PLGA)-poly(ethylene glycol) (PEG)-(PLGA); poly(lactic acid) (PLA)-(PEG)-(PLA); or poly(lactide-co-caprolactone) (PLCL)-(PEG)-(PLCL).

In one embodiment, the thermosensitive hydrogel comprises at least one polyurethane based polymer.

In one embodiment, the IL-2 polypeptide component is an IL-2 polypeptide, a recombinant IL-2 polypeptide, or an active IL-2 polypeptide fragment.

In one embodiment, the anti-IL-2 antibody component is JES6-1, JES6-1A12, S4B6, MQ1-17H12, 2F11C5, 2H20L7, C5, 8D5, BG5, OTI2C9, 5334, 5335, OT15A9, 419A7A3, AB12-3G4, 297C16G2, 297C16F11, 1C5, 1C1, 62, or 3C8.

In one embodiment, the composition comprises an IL-2 antigen:anti-IL-2 complex or a molecule resembling an IL-2 antigen:anti-IL-2 complex.

In one embodiment, the composition is administered as a low viscosity liquid.

In one embodiment, the composition is administered by way of injection.

In one embodiment, the disease or disorder is an autoimmune disease or disorder.

In one embodiment, the disease or disorder is rheumatoid arthritis (RA), type-1 diabetes (T1D), idiopathic inflammatory myopathies, multiple sclerosis, traumatic brain injury, wound healing, inflammatory diseases, pemphigus, psoriasis, psoriatic arthritis, systemic lupus erythematosus, ankylosing spondylitis, polymyositis, dermatomyositis, Sjogren's syndrome or different forms of vasculitis.

In one embodiment, the invention related to a kit comprising at least one thermosensitive hydrogel composition comprising:
a) at least one biologically compatible polymer; and
b) at least one of: an IL-2 polypeptide component, an anti-IL-2 antibody component and a combination thereof, wherein the composition is liquid at or below room temperature but forms a gel at about 37° C.

In one embodiment, the kit is formulated for storage at or below room temperature to maintain the thermostable hydrogel composition as a liquid.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A through FIG. 1C depict exemplary experimental data demonstrating that IL-2 gel inhibits autoimmune arthritis and mediates suppression of Th17 to Tfh cell conversion. FIG. 1A depicts data demonstrating that SFB− WT donor CD4⁺ fate-mapping T cells from KRN strain were transferred into SFB+ TCRα^{-/-}. BxN recipients (T cell deficient mice on a B6xNOD background and thus, bearing NOD MHC class II I-A^{g7} for self-Ag presentation). Recipient mice were treated with IL-2 gel or vehicle gel. Ankle thickening of different group recipient mice is shown as mean±SEM. Day 0 indicates the day of cell transfer. Sera were also collected 14 days after the adoptive transfer. Anti-GPI Ab titers are shown as mean±SEM (n=8-9/group, data combined from 2 independent assays). FIG. 1B depicts representative histogram plots and quantitative data of c-Maf expression in PP Rorγt+ T cells in mice from FIG. 1A. FIG. 1C depicts data demonstrating that splenocytes and PP cells from experiments in FIG. 1A were stained with Abs recognizing CD4, TCRβ, Foxp3, CXCR5 and PD-1. Representative plots and quantitative data of the percentage and number of ZsGreen+ and ZsGreen− Tfh cells are shown (n=8-9/group, data combined from 2 independent assays).

### DETAILED DESCRIPTION

The present invention relates generally to thermosensitive hydrogel compositions comprising IL-2 and anti-IL-2, where the hydrogel is liquid at low temperatures, but forms a gel at body temperature to provide for sustained delivery of IL-2 and anti-IL-2. The present invention further provides methods for use of the thermosensitive hydrogel compositions comprising IL-2 and anti-IL-2 for treating or preventing an autoimmune disease or disorder including, but not limited to, rheumatoid arthritis (RA), type-1 diabetes (T1D), idiopathic inflammatory myopathies, multiple sclerosis, traumatic brain injury, wound healing, inflammatory diseases, pemphigus, psoriasis, psoriatic arthritis, systemic lupus erythematosus, ankylosing spondylitis, polymyositis, dermatomyositis, Sjogren's syndrome and different forms of vasculitis.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations for example of ±20%, ±10%, ±5%, ±1%, and ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient. The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a drug delivery platform that temporally regulates drug release via selective thermosensitive hydrogel compositions comprising IL-2 and anti-IL-2 that can either be combined within a single hydrogel composition or added to independent hydrogels and subsequently mixed to provide for sustained delivery of IL-2 and anti-IL-2. The thermosensitive hydrogel compositions are liquid at or below room temperature, and then become a viscous hydrogel when exposed to tissues at body temperatures. Such a platform is ideal for the treatment of many diseases and disorders such as RA and other physiological conditions and diseases which would benefit from administration of IL-2 and anti-IL-2.

In accordance with one or more embodiments, the present invention provides compositions comprising IL-2, anti-IL-2 or a combination thereof incorporated into or dissolved within the thermosensitive hydrogel compositions. In some embodiments, the IL-2 and anti-IL-2 thermosensitive hydrogel compositions may further comprise at least one, two, or more addition biologically active agents dissolved within the hydrogel composition.

In some embodiments, the invention relates to compositions for sustained delivery of IL-2/anti-IL-2 monoclonal antibody complexes to subjects in need thereof. In some embodiments, the components of the IL-2/anti-IL-2 monoclonal antibody complexes (IL-2 protein and monoclonal anti-IL-2) are embedded separately in thermosensitive hydrogel compositions and combined before or during administration to a subject of interest. In some embodiments, the components of the IL-2/anti-IL-2 monoclonal antibody complexes (IL-2 protein and monoclonal anti-IL-2), or a molecule (e.g., a polypeptide) resembling or mimicking an IL-2 antigen:anti-IL-2 complex, are combined prior to being embedded into a single thermostable hydrogel composition.

### IL-2 Protein Compositions

The invention encompasses thermostable hydrogel compositions comprising an IL-2 polypeptide, a recombinant IL-2 polypeptide, an active IL-2 polypeptide fragment, or a IL-2 activator. To practice the methods of the invention; the skilled artisan would understand, based on the disclosure provided herein, how to formulate the appropriate IL-2 polypeptide, recombinant IL-2 polypeptide, active IL-2 polypeptide fragment, or IL-2 activator.

“Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, mutant polypeptides, variant polypeptides, or a combination thereof.

In one embodiment, the IL-2 polypeptide comprises the sequence: MYRMQLLSCIALSLALVTNSAPTSSSTKKTQLQLEHLLLDLQMILNGINNYKNPK LTRMLTFKFYMPKKATELKHLQCLEEELKPLEEVLNLAQSKNFHLRPRDLISNIN VIVLELKGSETTFMCEYADETATIVEFLNRWITFCQSIISTLT (SEQ ID NO:1) or a fragment or variant thereof.

“Variant” as used herein in reference to polypeptide molecules of the present invention, refers to a change in an amino acid sequence relative to a reference sequence and includes translocations, deletions, insertions, and substitutions/point mutations such as single nucleotide polymorphisms. For example, a variant may be 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to a reference amino acid sequence.

In some embodiments, the IL-2 protein or polypeptide comprises an amino acid sequence 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more identical to SEQ ID NO:1.

“Fragment” as used herein in reference to the polypeptides of the present invention, refers to a portion of a full-length amino acid sequence. For example, a fragment may be a polypeptide molecule that is 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the length of a reference amino acid sequence.

In some embodiments, the IL-2 protein or polypeptide comprises a fragment of SEQ ID NO:1 comprising 50% or more, 60% or more, 70% or more, 75% or more, 80% or more, 85% or more, 90% or more, 91% or more, 92% or more, 93% or more, 94% or more, 95% or more, 96% or more, 97% or more, 98% or more, or 99% or more of the full length sequence of SEQ ID NO:1.

Polypeptide(s) of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

The polypeptide(s) may alternatively be made by recombinant means or by cleavage from one or more longer polypeptide(s). The composition of the polypeptide(s) may be confirmed by amino acid analysis or sequencing.

The polypeptide(s) of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptide(s) of the invention may include unnatural amino acids formed by post-translational modification or by introducing unnatural amino acids during translation. A variety of approaches are available for introducing unnatural amino acids during protein translation.

The polypeptide(s) of the invention may be phosphorylated using conventional methods such as the method described in Reedijk et al. (The EMBO Journal 11(4):1365, 1992).

The polypeptide(s) of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Antibody Compositions

In some embodiments, the invention relates to compositions comprising at least one antibody, or fragment thereof, specific for binding to IL-2. In one embodiment, anti-IL-2 antibody is a neutralizing antibody. Exemplary anti-IL-2 antibodies that can be embedded in a thermostable hydrogel of the invention include, but are not limited to, JES6-1, JES6-1A12, S4B6, MQ1-17H12, 2F11C5, 2H20L7, C5, 8D5, BG5, OTI2C9, 5334, 5335, OTI5A9, 419A7A3, AB12-3G4, 297C16G2, 297C16F11, 1C5, 1C1, 62, and 3C8; or a fragment thereof.

As used herein, the term “antibody” or “immunoglobulin” refers to proteins (including glycoproteins) of the immunoglobulin (Ig) superfamily of proteins. An antibody or immunoglobulin (Ig) molecule may be tetrameric, comprising two identical light chain polypeptides and two identical heavy chain polypeptides. The two heavy chains are linked together by disulfide bonds, and each heavy chain is linked to a light chain by a disulfide bond. Each full-length Ig molecule contains at least two binding sites for a specific target or antigen.

An anti-IL-2 neutralizing or non-neutralizing antibody, or antigen-binding fragment thereof, includes, but is not limited to a polyclonal antibody, a monoclonal fusion proteins, antibodies or fragments thereof, chimerized or chimeric fusion proteins, antibodies or fragments thereof, humanized fusion proteins, antibodies or fragments thereof, deimmunized humfusion proteins, antibodies or fragments thereof, fully humfusion proteins, antibodies or fragments thereof, single chain antibody, single chain Fv fragment (scFv), Fv, Fd fragment, Fab fragment, Fab′ fragment, F(ab′)₂ fragment, diabody or antigen-binding fragment thereof, minibody or antigen-binding fragment thereof, triabody or antigen-binding fragment thereof, domain fusion proteins, antibodies or fragments thereof, camelid fusion proteins, antibodies or fragments thereof, dromedary fusion proteins, antibodies or fragments thereof, phage-displayed fusion proteins, antibodies or fragments thereof, or antibody, or antigen-binding fragment thereof, identified with a repetitive backbone array (e.g. repetitive antigen display).

As used throughout the present disclosure, the term “antibody” further refers to a whole or intact antibody (e.g., IgM, IgG, IgA, IgD, or IgE) molecule that is generated by any one of a variety of methods that are known in the art and described herein. The term “antibody” includes a polyclonal antibody, a monoclonal antibody, a chimerized or chimeric antibody, a humanized antibody, a deimmunized human antibody, and a fully human antibody. The antibody can be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice. The antibody can be a purified or a recombinant antibody.

### Thermosensitive Hydrogel Compositions

In accordance with an embodiment, the present invention provides a thermosensitive hydrogel composition comprising at least one, two, or more biologically compatible triblock copolymers and one or more biologically active agents, and wherein the composition having a gelation temperature dependent on the weight % ratio of the at least two or more triblock copolymers in said mixture.

As used herein, the term “triblock copolymers” means a biodegradable polymer of the formula A-block-B-block-A. Examples of such polymers useful in the composition of the present invention include, but are not limited to, poly(lactide-co-glycolide)-b-poly(ethylene glycol)-b-poly(lactide-co-glycolide) (PLGA)-(PEG)-(PLGA); (PEG)-(PLGA)-(PEG); poly(lactic acid) (PLA)-(PEG)-(PLA); (PEG)-(PLA)-(PEG); poly(lactide-co-caprolactone) (PLCL)-(PEG)-(PLCL); (PEG)-(PLCL)-(PEG); poly(caprolactone) (PCL)-(PEG)-(PCL); (PEG)-(PCL)-(PEG); poly(caprolactone)-b-poly(tetrahydrofuran)-b-poly(caprolactone) (PCL)-(PTHF)-(PCL); and poly(glycolide)-b-poly(ethylene glycol)-b-poly(glycolide) (PGA)-(PEG)-(PGA); (PEG)-(PGA)-(PEG).

A biologically compatible polymer refers to a polymer which is functionalized to serve as a composition for creating an injectable drug. The polymer is one that is a naturally occurring polymer or one that is not toxic to the host. The polymer may be a homopolymer where all monomers are the same or a hetereopolymer containing two or more kinds of monomers, such as triblock copolymers. The terms “biocompatible polymer,” “biocompatible cross-linked polymer matrix” and “biocompatibility” when used in relation to the instant polymers are art-recognized and are considered equivalent to one another, including to biologically compatible polymer. For example, biocompatible polymers include polymers that are neither toxic to the host (e.g., an animal or human), nor degrade (if the polymer degrades) at a rate that produces monomeric or oligomeric subunits or other byproducts at toxic concentrations in the host.

Polymer is used to refer to molecules composed of repeating monomer units, including homopolymers, block copolymers, heteropolymers, random copolymers, graft copolymers and so on. “Polymers” also include linear polymers as well as branched polymers, with branched polymers including highly branched, dendritic, and star polymers. A monomer is the basic repeating unit in a polymer. A monomer may itself be a monomer or may be dimer or oligomer of at least two different monomers, and each dimer or oligomer is repeated in a polymer.

Suitable polymers for the triblock copolymers useful in the thermosensitive hydrogels could include, but are not limited to, biocompatible monomers with recurring units found in poly(phosphoesters), poly(lactides), poly(glycolides), poly(caprolactones), poly(anhydrides), poly(amides), poly(urethanes), poly(esteramides), poly(orthoesters), poly(dioxanones), poly(acetals), poly(ketals), poly(carbonates), poly(orthocarbonates), poly(phosphazenes), poly(hydroxybutyrates), poly(hydroxyl valerates), poly(alkylene oxalates), poly(alkylene succinates), poly(malic acids), poly(amino acids), polyvinylalcohol, poly(vinylpyrrolidone), poly(ethylene glycol), poly(hydroxy cellulose), copolymers, terpolymers or combinations or mixtures of the above materials.

In some embodiments the weight % ratios of the mixture of two or more triblock copolymers can be in the range from 1/1, 2/1, 3/1, 3/2, 4/1, 4/3, 5/1, 5/2, 6/1, 7/1, 7/3, 8/1, 9/1 up to 10/1 depending on the gelation temperature desired. For example, a 3/1 ratio of PLGA-PEG-PLGA Mw 1,500: 1,500: 1,500 Da, 6: 1 LA:GA (86%/14% LA/GA) (w:w) PLGA-PEG-PLGA to PLGA-PEG-PLGA LG 50:50 (W:W) (MN −1,000: 1,000: 1,000 DA), when mixed together, provides a gelation temperature of 37° C.

In one embodiment, the hydrogel compositions of the invention are stable. As used herein, the terms “stability” and “stable” in the context of a liquid formulation comprising a biopolymer of interest that is resistant to thermal and chemical aggregation, degradation or fragmentation under given manufacture, preparation, transportation and storage conditions, such as, for one month, for two months, for three months, for four months, for five months, for six months or more. The “stable” formulations of the invention retain biological activity equal to or more than 80%, 85%, 90%, 95%, 98%, 99% or 99.5% under given manufacture, preparation, transportation and storage conditions. The stability of said preparation can be assessed by degrees of aggregation, degradation or fragmentation by methods known to those skilled in the art.

Biocompatible polymer and biocompatibility are art-recognized. For example, biocompatible polymers include polymers that are neither themselves toxic to the host (e.g., and animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art, using, for example, chemical means or enzymatic means. An aliquot of the treated sample products are placed in culture plates previously seeded with the cells. The sample products are incubated with the cells. The results of the assay may be plotted as % relative growth vs. concentration of degraded sample.

In addition, monomers, polymers, polymer matrices, and formulations of the present invention may also be evaluated by well-known in vivo tests, such as subcutaneous implantations or injections to rodents, rabbits, mini pigs, or non-human primates, etc. to confirm that they do not cause significant levels of irritation or inflammation at the implantation/injection sites.

“Biodegradable” is art-recognized, and includes monomers, polymers, polymer matrices, gels, compositions and formulations, such as those described herein, that are intended to degrade during use, such as in vivo. Biodegradable polymers and matrices typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain, functional group and so on to the polymer backbone. For example, a therapeutic agent, biologically active agent, or other chemical moiety attached as a side chain to the polymer backbone may be released by biodegradation. In certain embodiments, one or the other or both general types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible.”

In certain embodiments, the biodegradation rate of such polymer may be characterized by the presence of enzymes, for example, a chondroitinase. In such circumstances, the biodegradation rate may depend on not only the chemical identity and physical characteristics of the polymer matrix, but also on the identity of any such enzyme.

In certain embodiments, hydrogel polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between about 25 and 40° C. In other embodiments, the polymer degrades in a period of between about one hour and several weeks, depending on the desired application. In some embodiments, the polymer or polymer matrix may include a detectable agent that is released on degradation.

It will be understood by those of skill in the art, that the degradation or drug eluting properties of the hydrogels can be modified by adjusting the ratios of the two or more block copolymers used in the composition. For example, by blending the ratio (PLGA)-(PEG)-(PLGA), with a triblock copolymer of same type but different properties (e.g. molecular weight, lactic to glycolic acid ratio) or with (PLA)-(PEG)-(PLA) and/or (PLCL)-(PEG)-(PLCL), one can get a desired degradation and drug elution profile.

It will also be understood by those of skill in the art, that the amount of biologically active agent (e.g., IL-2, anti-IL-2 or a combination thereof) added during the manufacturing of the hydrogel can be varied as well. Typically, the amount of drug added can be in the range of 0.1 mg to about 100 mg, up to about 1,000 mg.

In some embodiments, a desired degradation and drug elution profile can be further altered by first encapsulating the drug or compound of interest in a microparticle comprised of a biodegradable and biocompatible polymer. The resulting microparticles containing the drug of interest can then be incorporated into the triblock copolymer composition and mixed and then administered to a subject.

Methods for the synthesis of the polymers described above are known to those skilled in the art, see, e.g., Concise Encyclopedia of Polymer Science and Polymeric Amines and Ammonium Salts, E. Goethals, editor (Pergamen Press, Elmsford, N.Y. 1980). Many polymers, such as PLGA, are commercially available. Naturally occurring polymers can be isolated from biological sources as known in the art or are commercially available. Naturally occurring and synthetic polymers may be modified using chemical reactions available in the art and described, for example, in March, “Advanced Organic Chemistry,” 4th Edition, 1992, Wiley-Interscience Publication, New York.

“Gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two-dimensional surface).

Hydrogels consist of hydrophilic polymers cross-linked to from a water-swollen, insoluble polymer network. Cross-linking can be initiated by many physical or chemical mechanisms. The hydrogels of interest also are configured to have a viscosity that will enable the gelled hydrogel to remain affixed on or in the cell, tissue or organ, or surface. Viscosity can be controlled by the monomers and polymers used, by the level of water trapped in the hydrogel, and by incorporated thickeners, such as biopolymers, such as proteins, lipids, saccharides and the like.

The terms “incorporated,” “encapsulated,”, “entrapped” and “impregnated” are art-recognized when used in reference to a therapeutic agent, dye, or other material and a polymeric composition, such as a composition of the present invention. In certain embodiments, these terms include incorporating, formulating or otherwise including such agent into a composition that allows for sustained release of such agent in the desired application. The terms may contemplate any manner by which a therapeutic agent or other material is incorporated into a polymer matrix, including, for example, attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved in the polymer. Any form of encapsulation or incorporation is contemplated by the present invention, in so much as the sustained release of any encapsulated therapeutic agent or other material determines whether the form of encapsulation is sufficiently acceptable for any particular use.

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

The terms “active agent” and “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed. In some embodiments of the invention, the active agent or biologically active agent is an IL-2 protein, an anti-IL-2 monoclonal antibody, or a combination thereof in the form of an IL-2:anti-IL-2 complex or a molecule (e.g., a polypeptide) resembling or mimicking an IL-2 antigen:anti-IL-2 complex.

Pharmaceutically acceptable salts are art-recognized, and include relatively nontoxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

Biodegradable polymers that can be incorporated into a thermostable hydrogel of the invention include, but are not limited to, polyglycolide, polylactide, poly (lactide-co-glycolide), polydioxanone, polycaprolactone, poly (trimethylene carbonate), poly (propylene fumarate), polyurethane, poly (ester amide), poly (Orthoester), Polyanhydride, Poly (amino acid), Polyphosphazene, Bacterial polyester.

In accordance with an embodiment, the present invention provides a thermosensitive hydrogel composition comprising: a) at least one, two, or more biologically compatible polymers selected from the group consisting of: polyglycolide, polylactide, poly (lactide-co-glycolide), polydioxanone, polycaprolactone, poly (trimethylene carbonate), poly (propylene fumarate), polyurethane, poly (ester amide), poly (Orthoester), Polyanhydride, Poly (amino acid), Polyphosphazene, and Bacterial polyester, and b) an IL-2 protein, an anti-IL-2 monoclonal antibody, or a combination thereof in the form of an IL-2:anti-IL-2 complex or a molecule (e.g., a polypeptide) resembling or mimicking an IL-2 antigen:anti-IL-2 complex; and wherein the composition having a gelation temperature dependent on the weight % ratio of the at least one, two, or more triblock copolymers in said mixture. In one embodiment, the thermosensitive hydrogel composition comprises polyurethane.

In accordance with an embodiment, the present invention provides a thermosensitive hydrogel composition comprising: a) at least one, two, or more biologically compatible triblock copolymers selected from the group consisting of: poly(D,L-lactide-co-glycolide) (PLGA)-poly(ethylene glycol) (PEG)-(PLGA); poly(lactic acid) (PLA)-(PEG)-(PLA); poly(lactide-co-caprolactone) (PLCL)-(PEG)-(PLCL), and b) an IL-2 protein, an anti-IL-2 monoclonal antibody, or a combination thereof in the form of an IL-2:anti-IL-2 complex or a molecule (e.g., a polypeptide) resembling or mimicking an IL-2 antigen:anti-IL-2 complex; and wherein the composition having a gelation temperature dependent on the weight % ratio of the at least one, two, or more triblock copolymers in said mixture.

It will be understood by those of skill in the art, that the biologically active agents can be incorporated into the same or different triblock copolymers of the composition, and can also be incorporated into a microparticle prior to being incorporated into the triblock copolymers of the inventive thermosensitive hydrogel.

It will be also understood by those of skill in the art, that an increase in the molecular weights of the polymers used in the triblock copolymer will increase the gelation temperature of the composition.

In certain embodiments, the subject compositions comprise about 1% to about 75% or more by weight of the total composition, alternatively about 2.5%, 5%, 10%, 20%, 30%, 40%, 50%, 60% or 70%, of the biologically active agent.

Buffers, acids and bases may be incorporated in the compositions to adjust pH. Agents to increase the diffusion distance of agents released from the composition may also be included.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

Buffering agents help to maintain the pH in the range which approximates physiological conditions. In some embodiments, buffers are present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

In some embodiments, isotonicifiers are present to ensure physiological isotonicity of liquid compositions of the instant invention and include polyhydric sugar alcohols, for example, trihydric or higher sugar alcohols, such as glycerin, erythritol, arabitol, xylitol, sorbitol and mannitol. Polyhydric alcohols can be present in an amount of between about 0.1% to about 25%, by weight, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of biopolymer.

Additional miscellaneous excipients include bulking agents, (e.g., starch), chelating agents (e.g., EDTA), antioxidants (e.g., ascorbic acid, methionine or vitamin E) and cosolvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml.

The instant invention encompasses formulations, such as, liquid formulations having stability at temperatures found in a commercial refrigerator and freezer found in the office of a physician or laboratory, such as from about 20° C. to about 2° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours or more prior to use.

The formulations to be used for in vivo administration must be sterile. That can be accomplished, for example, by filtration through sterile filtration membranes. For example, the formulations of the present invention may be sterilized by filtration.

The thermosensitive hydrogel compositions will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the biopolymer to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease.

In another embodiment, an effective amount of a therapeutic or a prophylactic agent of interest reduces the symptoms of a disease by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Also used herein as an equivalent is the term, “therapeutically effective amount.”

As used herein, the term “treat,” as well as words stemming there from, includes preventative as well as disorder remitative treatment. The terms “reduce,” “suppress,” “prevent,” and “inhibit,” as well as words stemming there from, have their commonly understood meaning of lessening or decreasing. These words do not necessarily imply 100% or complete treatment, reduction, suppression, or inhibition.

Embodiments of the invention also include a process for preparing pharmaceutical products comprising the inventive thermosensitive hydrogel compositions. The term “pharmaceutical product” means a composition suitable for pharmaceutical use (pharmaceutical composition), as defined herein. Pharmaceutical compositions formulated for particular applications comprising the compounds of the present invention are also part of this invention, and are to be considered an embodiment thereof. Examples of such products can include hydrogel compositions incorporating drugs or other biologically active agents, and also the hydrogel compositions comprising microparticles having drugs or other biologically active agents encapsulated within the microparticles in the hydrogel composition.

### Methods

The invention also provides methods and systems for administering the thermostable hydrogel compositions comprising an IL-2 protein an anti-IL-2 antibody or a combination thereof of the invention may be used to treat, ameliorate, inhibit, or prevent an autoimmune disease or disorder. Exemplary diseases or disorders that can be treated or prevented using the thermostable hydrogel compositions of the invention include, but are not limited to, rheumatoid arthritis (RA), type-1 diabetes (T1D), idiopathic inflammatory myopathies, multiple sclerosis, traumatic brain injury, wound healing, inflammatory diseases, pemphigus, psoriasis, psoriatic arthritis, systemic lupus erythematosus, ankylosing spondylitis, polymyositis, dermatomyositis, Sjogren's syndrome and different forms of vasculitis.

In one embodiment, the present invention provides a method comprising the thermostable hydrogel compositions comprising an IL-2 protein, an anti-IL-2 antibody or a combination thereof to a subject having an autoimmune disease or disorder.

In an embodiment, the term “administering” means that the compounds of the present invention are introduced into a subject, for example a subject diagnosed with an autoimmune disease or disorder, using one or more known routes of administration. In some embodiments, the compositions of the invention are administrated by way of injection.

In one embodiment, one or more thermostable hydrogel of the invention is co-administered with one or more additional hydrogel composition or one or more additional therapeutic agent or adjuvant. “Co-administration” as used herein is understood as administration of one or more agents to a subject such that the agents are present and active in the subject at the same time. Co-administration does not require a preparation of an admixture of the agents or simultaneous administration of the agents.

One of skill in the art will appreciate that a thermostable hydrogel comprising an IL-2 polypeptide, a recombinant IL-2 polypeptide, or an active IL-2 polypeptide fragment and a thermostable hydrogel comprising an anti-IL-2 antibody can be administered singly or in any combination thereof. Further, a thermostable hydrogel comprising an IL-2 polypeptide, a recombinant IL-2 polypeptide, or an active IL-2 polypeptide fragment and a thermostable hydrogel comprising an anti-IL-2 antibody can be administered singly or in any combination thereof in a temporal sense, in that they may be administered simultaneously, before, and/or after each other. One of ordinary skill in the art will appreciate, based on the disclosure provided herein, that a thermostable hydrogel comprising an IL-2 polypeptide, a recombinant IL-2 polypeptide, or an active IL-2 polypeptide fragment and a thermostable hydrogel comprising an anti-IL-2 antibody can be used to prevent or treat an autoimmune disease or disorder.

The optimal effective amount of the compositions can be determined empirically and will depend on the type and severity of the disease, route of administration, disease progression and health, mass and body area of the individual. Such determinations are within the skill of one in the art. The effective amount can also be determined based on in vitro complement activation assays. Examples of dosages of molecules which can be used for methods described herein include, but are not limited to, an effective amount within the dosage range of any of about 0.01 mg/kg to about 300 mg/kg, or within about 0.1 mg/kg to about 40 mg/kg, or with about 1 mg/kg to about 20 mg/kg, or within about 1 mg/kg to about 10 mg/kg. In some embodiments, the amount of composition administered to an individual is about 10 mg to about 500 mg per dose, including for example any of about 10 mg to about 50 mg, about 50 mg to about 100 mg, about 100 mg to about 200 mg, about 200 mg to about 300 mg, about 300 mg to about 500 mg, about 500 mg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per dose.

The compositions may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgical implantation in various locations.

Dosage amounts and frequency will vary according the particular formulation, the dosage form, and individual patient characteristics. Generally speaking, determining the dosage amount and frequency for a particular formulation, dosage form, and individual patient characteristic can be accomplished using conventional dosing studies, coupled with appropriate diagnostics.

### Unit Dosages, Articles of Manufacture, and Kits

Also provided are unit dosage forms of compositions, each dosage containing from about 0.01 mg to about 50 mg, including for example any of about 0.1 mg to about 50 mg, about 1 mg to about 50 mg, about 5 mg to about 40 mg, about 10 mg to about 20 mg, or about 15 mg of the targeted molecule. In some embodiments, the unit dosage forms of targeted molecule composition comprise about any of 0.01 mg-0.1 mg, 0.1 mg-0.2 mg, 0.2 mg-0.25 mg, 0.25 mg-0.3 mg, 0.3 mg-0.35 mg, 0.35 mg-0.4 mg, 0.4 mg-0.5 mg, 0.5 mg-1.0 mg, 10 mg-20 mg, 20 mg-50 mg, 50 mg-80 mg, 80 mg-100 mg, 100 mg-150 mg, 150 mg-200 mg, 200 mg-250 mg, 250 mg-300 mg, 300 mg-400 mg, or 400 mg-500 mg targeted inhibitor molecule. In some embodiments, the unit dosage form comprises about 0.25 mg targeted molecule. The term “unit dosage form” refers to a physically discrete unit suitable as unitary dosages for an individual, each unit containing a predetermined quantity of active material calculated to produce the desired therapeutic effect, in association with a suitable pharmaceutical carrier, diluent, or excipient. These unit dosage forms can be stored in suitable packaging in single or multiple unit dosages and may also be further sterilized and sealed.

The present invention also provides kits comprising compositions (or unit dosages forms and/or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

In one embodiment, an article of manufacture containing thermosensitive hydrogel compositions useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating, for example, an autoimmune disease and may have a sterile access port (for example, the container may be a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including buffers, diluents, filters, needles, syringes and package inserts with instructions for use.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, point out specific embodiments of the present invention, and are not to be construed as limiting in any way the remainder of the disclosure.

### Example 1: Thermoresponsive Hydrogels

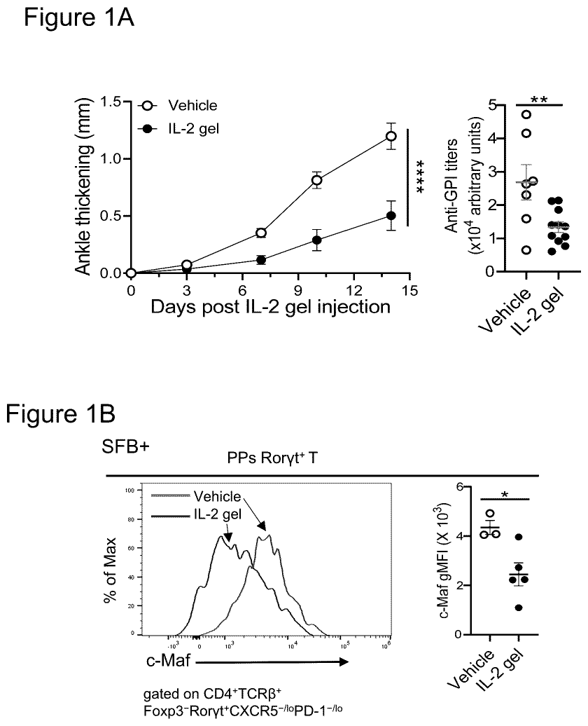

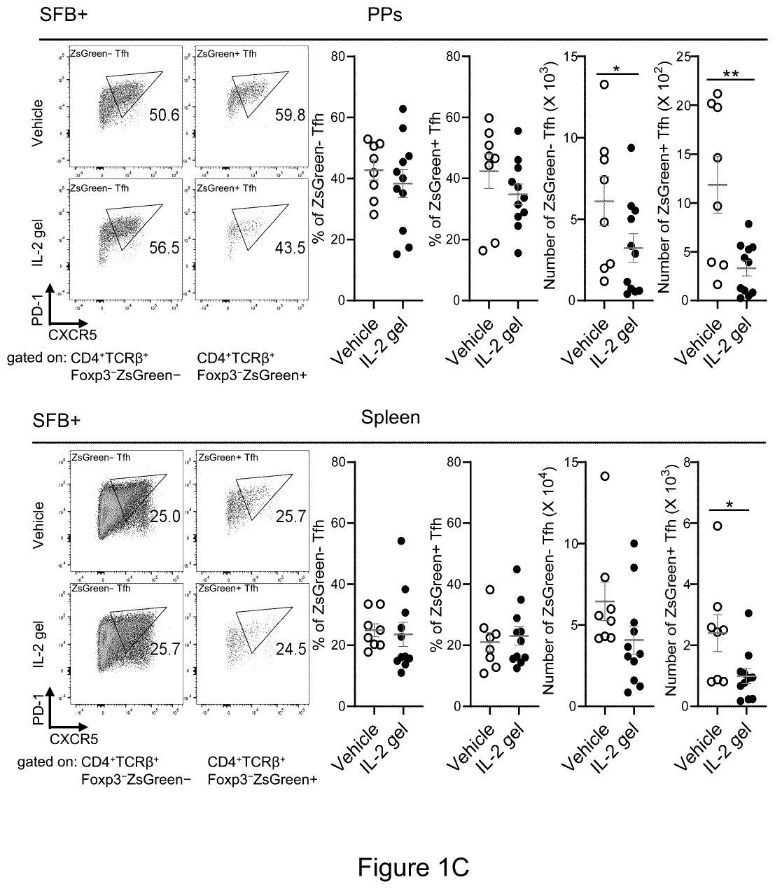

Thermoresponsive hydrogels (liquid at 4° C. and gel at 37° C.) were generated that can release IL-2 and mAb-IL-2 complex (FIG. 1A and FIG. 1B).

The materials used are: Mouse recombinant IL-12 (catalog number: 10788-764, VWR Inc., Pittsburgh, PA), monoclonal IL-2 antibody (catalog number: 103572-482, MAB RAT IL-2 JES6-1A12 MOUSE IGG2A K, VWR Inc., Pittsburgh, PA), Poly(lactide-co-glycolide)-b-poly(ethylene glycol)-b-poly(lactide-co-glycolide)-PLGA-PEG-PLGA (catalog number: NC1632817, Fisher Scientific, Waltham, MA).

To generate the thermoresponsive hydrogels, first 200 mg/mL poly(lactide-co-glycolide)-b-poly(ethylene glycol)-b-poly(lactide-co-glycolide) (PLGA-PEG-PLGA) solution was generated in 1×PBS by vortexing the mixture for 16 hours at 4° C. Next, 200 μg of mAb-IL-2 (stock concentration 8.9 mg/mL) is added to the 200 μg of IL-2 (powder form) and incubated for 15 minutes at 4° C. To the same tube, 500 μL of PLGA-PEG-PLGA solution in PBS at 4° C. was added, and the solution was gently pipetted. To ensure that the mixture remains in liquid form for ease of injection through syringe, at all times all the solutions were maintained at 4° C. Moreover, at body temperature of 37° C. (e.g. after injection in the body) the mixture forms a hydrogel. The solution thus generated were frozen and stored at −80° C. until used. This solution was generated for 5 mice, so that with one-time injection, each mouse received 16 μg of IL-2 and 16 μg of mAb-IL-2.

FIG. 1A through FIG. 1C demonstrate that IL-2 gel inhibits autoimmune arthritis and mediates suppression of Th17 to Tfh cell conversion. FIG. 1A depicts data demonstrating that SFB− WT donor CD4⁺ fate-mapping T cells from KRN strain were transferred into SFB+ TCRα^{-/-}.BxN recipients (T cell deficient mice on a B6xNOD background and thus, bearing NOD MHC class II I-A^{g7} for self-Ag presentation). Recipient mice were treated with IL-2 gel or vehicle gel. Ankle thickening of different group recipient mice is shown as mean±SEM. Day 0 indicates the day of cell transfer. Sera were also collected 14 days after the adoptive transfer. Anti-GPI Ab titers are shown as mean±SEM (n=8-9/group, data combined from 2 independent assays).

FIG. 1B depicts representative histogram plots and quantitative data of c-Maf expression in PP Rorγt+ T cells in mice from FIG. 1A.

FIG. 1C depicts data demonstrating that splenocytes and PP cells from experiments in FIG. 1A were stained with Abs recognizing CD4, TCRβ, Foxp3, CXCR5 and PD-1. Representative plots and quantitative data of the percentage and number of ZsGreen+ and ZsGreen− Tfh cells are shown (n=8-9/group, data combined from 2 independent assays). It was observed that this dose of the IL-2 gel provided little or no toxicity to CD4+ T cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A thermosensitive hydrogel composition comprising:
a) at least one biologically compatible polymer; and
b) at least one biologically active agent selected from the group consisting of: an IL-2 polypeptide component, an anti-IL-2 antibody component and a combination thereof; and wherein the composition is liquid at or below room temperature but forms a gel at about 37° C.

2. The thermosensitive hydrogel of claim 1, comprising at least one biologically compatible polymer selected from the group consisting of: poly(D,L-lactide-co-glycolide) (PLGA)-poly(ethylene glycol) (PEG)-(PLGA); poly(lactic acid) (PLA)-(PEG)-(PLA); and poly(lactide-co-caprolactone) (PLCL)-(PEG)-(PLCL).

3. The thermosensitive hydrogel of claim 1, comprising at least one polyurethane based polymer.

4. The thermosensitive hydrogel composition of claim 1, wherein the IL-2 polypeptide component is selected from the group consisting of IL-2 polypeptide, a recombinant IL-2 polypeptide, and an active IL-2 polypeptide fragment.

5. The thermosensitive hydrogel composition of claim 1, wherein the composition comprises a combination of an IL-2 polypeptide and an anti-IL-2 antibody.

6. The thermosensitive hydrogel composition of claim 1, comprising an IL-2 antigen:anti-IL-2 complex or a molecule resembling an IL-2 antigen:anti-IL-2 complex.

7. The thermosensitive hydrogel composition of claim 1, wherein the composition is a low viscosity liquid at room temperature.

8. A method of treating a disease or disorder, the method comprising administering an effective amount of at least one thermosensitive hydrogel of claim 1 to a subject in need thereof.

9. The method of claim 8, wherein the thermosensitive hydrogel comprises at least one biologically compatible polymer selected from the group consisting of: poly(D,L-lactide-co-glycolide) (PLGA)-poly(ethylene glycol) (PEG)-(PLGA); poly(lactic acid) (PLA)-(PEG)-(PLA); and poly(lactide-co-caprolactone) (PLCL)-(PEG)-(PLCL).

10. The method of claim 8, comprising at least one polyurethane based polymer.

11. The method of claim 8, wherein the IL-2 polypeptide component is selected from the group consisting of IL-2 polypeptide, a recombinant IL-2 polypeptide, and an active IL-2 polypeptide fragment.

12. The method of claim 8, comprising an IL-2 antigen:anti-IL-2 complex or a molecule resembling an IL-2 antigen:anti-IL-2 complex.

13. The method of claim 8, wherein the composition is administered as a low viscosity liquid.

14. The method of claim 8, wherein the composition is administered by way of injection.

15. The method of claim 8, wherein the disease or disorder is an autoimmune disease or disorder.

16. The method of claim 8, wherein the disease or disorder is selected from the group consisting of rheumatoid arthritis (RA), type-1 diabetes (T1D), idiopathic inflammatory myopathies, multiple sclerosis, traumatic brain injury, wound healing, inflammatory diseases, pemphigus, psoriasis, psoriatic arthritis, systemic lupus erythematosus, ankylosing spondylitis, polymyositis, dermatomyositis, Sjogren's syndrome and different forms of vasculitis.

17. A kit comprising at least one thermostable hydrogel of claim 1.

18. The kit of claim 17, wherein the kit is formulated for storage at or below room temperature to maintain the thermostable hydrogel composition as a liquid.


SEQUENCE LISTING (1 sequence)

SEQ ID NO: 1 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT